(19) United States
(12) Patent Application Publication
(10) Pub. No.: US 2024/0257955 A1
(43) Pub. Date: Aug. 1, 2024

(54) Aid for Medical Care Dispatch

(71) Applicant: instED, LLC, Boston, MA (US)

(72) Inventors: Mohsin Khan, Northborough, MA (US); Karin Falkenberg, Raymond, ME (US); Jennifer Femino, Beverly, MA (US); Ismail Deif, Concord, NC (US); Dan Henderson, Brookline, MA (US); Sebastiaan Foppema, Cumberland, RI (US); Aijaz Baloch, Newton, MA (US)

(21) Appl. No.: 18/162,316

(22) Filed: Jan. 31, 2023

(51) Int. Cl. G16H 40/20; G16H 10/60; G16H 50/30

(52) U.S. Cl. CPC G16H 40/20; G16H 10/60; G16H 50/30

(57) ABSTRACT

Techniques are provided for assisting with medical service dispatch by providing recommendations to a clinical resource coordinator and by predicting upcoming demand for medical services. An example method includes receiving a request for a patient to receive medical care, determining if the patient is experiencing a medical emergency based at least in part on the patient symptoms and the patient personal medical history, recommending that the patient seek emergency medical services in response to determining that the request contains at least one critical symptom or determining that a sum of weighted risk values exceeds the threshold value, and in response to determining the request does not contain at least one critical symptom and the sum of the weighted risk values does not exceed the threshold value, recommending a medical care provider to visit the patient based at least in part on the availability information of the medical care provider.

### BACKGROUND

Persons in need of medical care often seek such medical care at a hospital's emergency department (ED) and/or a corresponding emergency room (ER), even if emergency medical care is not necessary. This may result in an undesirable use of important hospital resources. Additionally, persons seeking medical care may have difficulty traveling to a hospital or urgent care clinic and may have to depend on ambulance services, which may be unnecessary and expensive. These persons may be better served by an at-home visit performed by one or more medical professionals, such as Emergency Medical Technicians (EMTs), paramedics, or other such trained professionals. Services that coordinate and dispatch medical professionals to patient homes would benefit from tools and processes that improve their communication and efficiency.

### SUMMARY

An example method for assisting with medical care dispatch according to the disclosure includes receiving a request for a patient to receive medical care including, at least, patient symptoms and patient personal medical history, receiving availability information of at least one medical care provider, determining if the patient is experiencing a medical emergency based at least in part on the patient symptoms and the patient personal medical history, such that the determining if the patient is experiencing the medical emergency includes parsing the request, providing one or more elements of the parsed request to a machine learning model, determining if the request contains at least one critical symptom which corresponds to a critical illness based on an output of the machine learning model, assigning a weighted risk value to one or more general symptoms based on the output of the machine learning model, and determining if a sum of the weighted risk values for the one or more general symptoms exceeds a threshold value, recommending that the patient seek emergency medical services in response to determining that the request contains at least one critical symptom or determining that the sum of the weighted risk values exceeds the threshold value, and in response to determining the request does not contain at least one critical symptom and the sum of the weighted risk values does not exceed the threshold value, recommending a medical care provider to visit the patient based at least in part on the availability information of the medical care provider.

Implementations of such a method may include one or more of the following features. A confidence score associated with a recommendation that the medical care provider visit the patient may be determined. Expertise information of at least one medical care provider may be received. Recommending the medical care provider may be based at least in part on the expertise information of the medical care provider. The availability information may include cost information. Recommending that the patient seek emergency medical service may include contacting the emergency medical services. The request may be received via a patient portal executing in a web browser. The availability information may be received via a provider portal executing in a web browser. The one or more elements of the parsed request may include at least one of a location, a symptom, a medical history information, an indication of allergies, an indication of pain, and demographic information.

An example system for assisting with medical care dispatch according to the disclosure may include at least one server including a memory and at least one processor communicatively coupled to the memory and configured to: receive a request for a patient to receive medical care including, at least, patient symptoms and patient personal medical history, receive availability information of at least one medical care provider, determine if the patient is experiencing a medical emergency based at least in part on the patient symptoms and the patient personal medical history, wherein the determining if the patient is experiencing the medical emergency includes parsing the request, providing one or more elements of the parsed request to a machine learning model, determining if the request contains at least one critical symptom which corresponds to a critical illness based on an output of the machine learning model, assigning a weighted risk value to one or more general symptoms based on the output of the machine learning model, and determining if a sum of the weighted risk values for the one or more general symptoms exceeds a threshold value, recommend that the patient seek emergency medical services in response to determining that the request contains at least one critical symptom or determining that the sum of the weighted risk values exceeds the threshold value, and in response to determining the request does not contain at least one critical symptom and the sum of the weighted risk values does not exceed the threshold value, recommend a medical care provider to visit the patient based at least in part on the availability information of the medical care provider.

### BRIEF DESCRIPTION OF THE DRAWINGS

FIG. 1 is a diagram of a medical resource management system, including a server and various users who communicate with the server via devices.

FIG. 2 is a block diagram of an embodiment of a server.

FIG. 3 is a block flow diagram of the steps of an example in-home medical service visit.

FIG. 4 is a block flow diagram of the creation and input of a medical service request.

FIG. 5 is a block flow diagram of an example decision support engine.

FIG. 6 shows an example user interface of a medical service visit request intake form.

FIG. 7 shows an example user interface of a medical service visit request assessment form.

FIG. 8 shows a block flow diagram of an example visit prediction engine.

FIG. 9 is a block flow diagram of an embodiment of an example algorithm used by the decision support engine of FIG. 5.

FIG. 10 is a block flow diagram of an embodiment of another example algorithm used by the decision support engine of FIG. 5.

FIG. 11 is a block flow diagram of a method of assisting with medical care dispatch.

FIG. 12 is a block flow diagram of a method of anticipating demand for medical services.

### DETAILED DESCRIPTION

Techniques are discussed herein for assisting with medical service coordination and dispatch by providing recommendations to a clinical resource coordinator and by predicting upcoming demand for medical services. For example, a medical care service request is received from a patient or provider and parsed by a server, and a recommendation for performing a medical service visit, for declining to perform a medical service visit, or for sending the patient to an emergency department (ED) is provided by the server. The server may also assist clinical resource coordinators to more effectively allocate resources by providing predictions of upcoming medical service demand. These techniques are examples only, and not exhaustive.

Items and/or techniques described herein may provide one or more of the following capabilities, as well as other capabilities not mentioned. A server may assist with receiving, assessing, and triaging patient requests for medical services. The server may assist with deciding whether to provide an in-home medical care visit to a patient, decline such a visit, or to send the patient to the ED. The server may assist with assigning medical providers and resources to a patient to handle the request. The server may also predict demand for medical services and recommend an appropriate supply and placement of medical resources. Other capabilities may be provided and not every implementation according to the disclosure must provide any, let alone all, of the capabilities discussed. Further, it may be possible for an effect noted above to be achieved by means other than that noted, and a noted item/technique may not necessarily yield the noted effect.

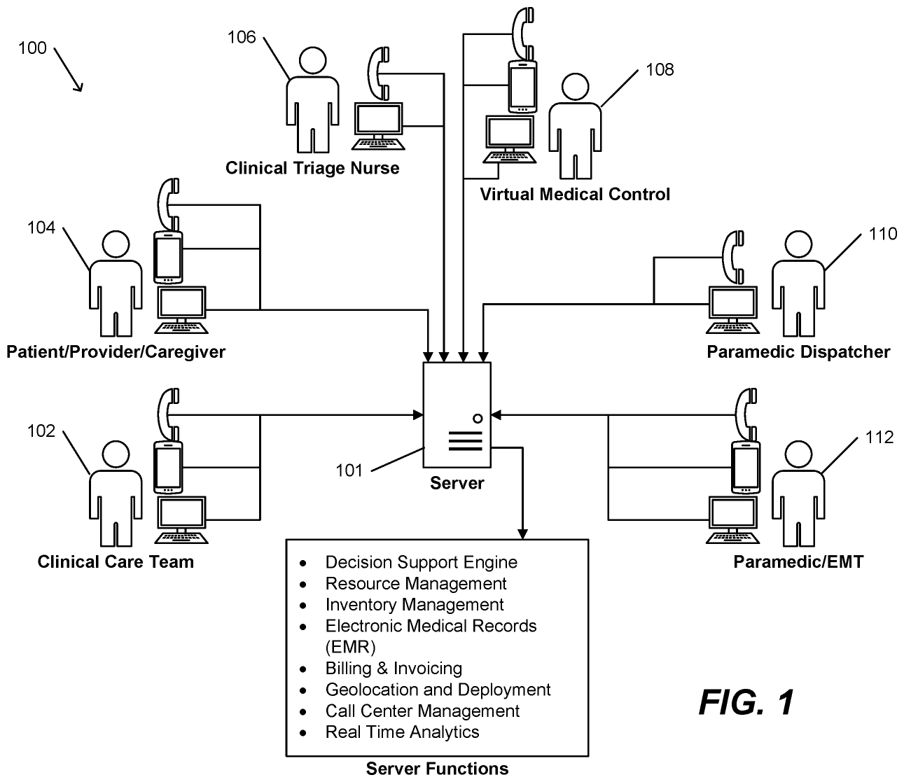

Referring to FIG. 1, an example medical resource management system 100 is shown. The medical resource management system 100 may comprise one or more servers such as the server 101. The server 101 may be one or more computers or other devices, and, if multiple devices, may be located at a single location or distributed across multiple locations. The server 101 may be one or more networked computers and may be configured to communicate via one or more network and Internet protocols (e.g., Ethernet, transmission control protocol/internet protocol (TCP/IP), point to point protocol (PPP), etc.). The server 101 may be provided by a cloud infrastructure service such as Amazon AWS, Microsoft Azure, or similar. The server 101 may be configured to communicate with one or more users of networked stations and applications such as a clinical care team 102; a patient, a patient's medical provider (which may be different from the paramedic/VMC who may also be referred to herein as medical providers), and/or a patient's caregiver 104; a clinical triage nurse 106; a virtual medical control doctor 108; a paramedic dispatcher 110; and/or a paramedic/EMT 112. The server 101 may also be configured to communicate with other servers, systems, or computers, including those storing electronic medical records (EMR). The server 101 may also be configured to communicate with individuals other than those listed above and shown in FIG. 1. The users 102-112 may communicate with the server 101 via a computer (including a desktop, laptop, or similar), a mobile device (including a smartphone, tablet, or similar), a telephone (including a smartphone, a landline phone, a cellular phone, or similar), or other wired and wireless devices configured to communicate via one or more network connections. The server may comprise one or more user interfaces through which users 102-112 may interact and communicate with the server. The users 102-112 may utilize web-based applications configured to execute in a web-browser and/or an application executing on a computer or mobile device to communicate with the server 101.

The server 101 may comprise or be configured to perform the functions described herein. For example, the server 101 may be configured to perform the functions of: a decision support engine; resource management; inventory management; electronic medical records (EMR) processing and storage; billing & invoicing processing and storage; geolocation and deployment of medical resources; call center management; real-time analytics of medical requests and services, and system operations. In operation, the server 101 may be one or more applications executing in a cloud environment, and the users 102-112 may access the server 101 via a connection to the Internet.

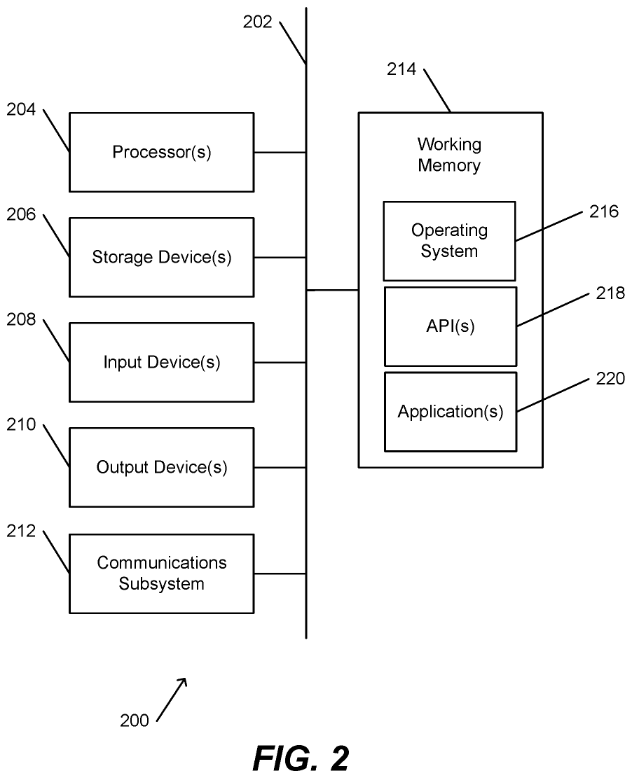

Referring to FIG. 2, an example computer system 200 is shown. The computer system 200 as illustrated in FIG. 2 may incorporate as part of the previously described computerized devices, including the server 101. FIG. 2 provides a schematic illustration of one embodiment of the computer system 200 that can perform the methods provided by various other embodiments, as described herein, and/or can function as the host computer system, or other networked computer system. In an example, the computer system 200 may be included in a cloud environment such as the Microsoft Azure platform and the operations and functions described herein may be distributed over different computer systems 200 operating in different locations. It should be noted that FIG. 2 is meant only to provide a generalized illustration of various components, any or all of which may be utilized as appropriate. FIG. 2, therefore, broadly illustrates how individual system elements may be implemented in a relatively separated or relatively more integrated manner.

The computer system 200 is shown comprising hardware elements that can be electrically coupled via a bus 202 (or may otherwise be in communication, as appropriate). In an example, the bus 202 may be configured as one or more communication channels in a cloud-computing environment. The hardware elements may include one or more processors 204, including without limitation one or more general-purpose processors and/or one or more special-purpose processors (such as digital signal processing chips, graphics acceleration processors, and/or the like); one or more input devices 208, which can include without limitation a mouse, a keyboard, a touchscreen and/or the like; and one or more output devices 210, which can include without limitation a display device, a printer and/or the like.

The computer system 200 may further include (and/or be in communication with) one or more non-transitory storage devices 206, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable and/or the like. Such storage devices may be configured to implement any appropriate data stores, including without limitation, various file systems, database structures, and/or the like.

The computer system 200 might also include a communications subsystem 212, which can include without limitation a modem, a network card (wireless or wired), an infrared communication device, a wireless communication device and/or chipset (such as a Bluetooth® device, an 802.11 device, a WiFi device, a WiMax device, cellular communication facilities, etc.), and/or the like. The communications subsystem 212 may permit data to be exchanged with a network (such as the network described below, to name one example), other computer systems, and/or any other devices described herein. In many embodiments, the computer system 200 will further comprise a working memory 214, which can include a RAM or ROM device, as described above.

The computer system 200 also can comprise software elements, shown as being currently located within the working memory 214, including an operating system 216, device drivers, executable libraries, and/or other code, such as one or more application programs 220, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods. In a cloud computing implementation, the working memory may include one or more application programming interfaces (APIs) 218 configured to send and receive data and instructions to and from other networked stations. For example, the users 102-112 may send and receive information to the server 101 via applications configured to utilize the APIs 218.

A set of these instructions and/or code might be stored on a computer-readable storage medium, such as the storage device(s) 206 described above. In some cases, the storage medium might be incorporated within a computer system, such as the system 200. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computer system 200 and/or might take the form of source and/or installable code, which, upon compilation and/or installation on the computer system 200 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer system 200) to perform methods in accordance with various embodiments of the disclosure. According to a set of embodiments, some or all of the procedures of such methods are performed by the computer system 200 in response to processor 204 executing one or more sequences of one or more instructions (which might be incorporated into the operating system 216 and/or other code, such as an application program 220) contained in the working memory 214. Such instructions may be read into the working memory 214 from another computer-readable medium, such as one or more of the storage device(s) 206. Merely by way of example, execution of the sequences of instructions contained in the working memory 214 might cause the processor(s) 204 to perform one or more procedures of the methods described herein.

The terms “machine-readable medium” and “computer-readable medium,” as used herein, refer to any medium that participates in providing data that causes a machine to operate in a specific fashion. In an embodiment implemented using the computer system 200, various computer-readable media might be involved in providing instructions/code to processor(s) 204 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 206. Volatile media include, without limitation, dynamic memory, such as the working memory 214. Transmission media include, without limitation, coaxial cables, copper wire and fiber optics, including the wires that comprise the bus 202, as well as the various components of the communication subsystem 212 (and/or the media by which the communications subsystem 212 provides communication with other devices).

Various forms of computer-readable media may be involved in carrying one or more sequences of one or more instructions to the processor(s) 204 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 200.

The communications subsystem 212 (and/or components thereof) generally will receive the signals, and the bus 202 then might carry the signals (and/or the data, instructions, etc. carried by the signals) to the working memory 214, from which the processor(s) 204 retrieves and executes the instructions. The instructions received by the working memory 214 may optionally be stored on a storage device 206 either before or after execution by the processor(s) 202.

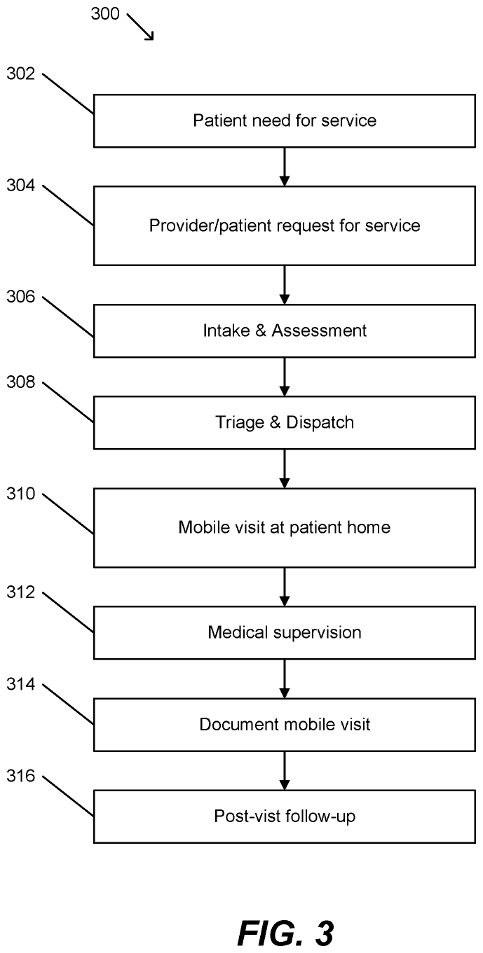

Referring to FIG. 3, an example of a life cycle 300 of a medical service request and visit is shown. The life cycle 300 begins at the first stage 302 wherein a patient experiences a need for medical services and, optionally, reaches out to a caregiver or other medical professional for assistance. In general, the stages in the life cycle 300 are examples, and not limitations. The life cycle 300 may be altered, e.g., by having stages added, removed, rearranged, combined, performed concurrently, and/or having single stages split into multiple stages. At stage 304, the patient, a medical provider for the patient, or a caregiver for the patient, creates and submits a request for medical services. At stage 306, the request, including intake information, is received and the request is assessed.

This assessment may be performed by a user (including, for example, the Clinical Care Team 102, the Clinical Triage Nurse 106, or the Virtual Medical Control 108) or a system (including, for example, the server 101), in whole or in part, and may include information associated with one or more assessments associated with the patient and/or the services to be provided. For example, the one or more assessments may include an urgency assessment, a diagnostic possibility assessment, a location and navigation assessment, a logistical assessment, a treatment capability assessment, a resource availability assessment, an insurance coverage assessment, a cost assessment, and a ROI assessment. Other patient and/or service related assessments may also be performed.

The urgency assessment may include determining, based on the information in the request, the level of importance and time-sensitivity for the patient to receive medical treatment. The diagnostic possibility assessment may include determining, based on the information in the request, one or more possible diagnoses that correspond to the symptoms being experienced by the patient. These diagnostic possibilities may inform other assessments such as the urgency assessment, the treatment capability assessment and others. The location and navigation assessment may include determining whether the patient is in a served area, the route to the patient, and the time it will take for a paramedic to reach them, based on the information in the request and, one or more of location information about medical resources (such as paramedics), traffic information, road condition information, and weather information. The logistical assessment may include determining, based on, at least, information in the request and other assessment information, such as the location and navigation assessment information, the logistical impact of sending a medical resource to the patient. The treatment capability assessment may include determining, based at least in part on information in the request and information about the medical resources available (including, e.g., competency of available paramedics, nurses, and/or doctors), whether the patient can be competently, thoroughly, and successfully treated. The resource availability assessment may include determining the quantity of resources available, the types of resources available, and the competency of medical professionals, including paramedics, doctors, and nurses. The insurance coverage assessment may include determining, based at least in part on information in the request, whether the patient has insurance, whether the insurance will cover treatment by the available medical professionals, and whether the insurance will cover treatment for the patient's condition(s). The cost assessment may include determining, based at least in part on information in the request and other assessment information, the cost of providing medical services to the patient. ROI assessment may include determining, based at least in part on information in the request and other assessment information, the financial benefit of providing medical services to the patient.

At stage 308, the request is triaged, and one or more medical professionals may be selected to serve the patient, typically including a paramedic 112 and a VMC 108, and the paramedic is dispatched by a paramedic dispatcher 110.

At stage 310, paramedics may visit the patient at the patient's home or current location to provide medical treatment to the patient. The medical treatment may be supervised, as indicated at stage 312, by the Virtual Medical Controller (VMC). The VMC may typically be a doctor or nurse practitioner. The medical supervision may involve the VMC and the paramedic, and optionally the patient, communicating with one another. The medical supervision may involve communication between any of the users 102-112 shown in FIG. 1. At stage 314, at least one of the paramedic, the VMC, and another user may document aspects and associated data from the mobile visit. The documentation may typically result in entry into electronic medical records (EMR). The documentation may also include post-visit urgency labelling by the paramedic or the VMC, which may or may not align with the urgency labeling provided by the patient's caregiver 104 or the clinical triage nurse 106. This documentation may be used as an input to one or more ML models as described herein. At stage 316, there may be a post-visit follow-up for the patient scheduled. This may involve another mobile visit at the patient's home, or may be a virtual follow-up, or may involve the patient traveling to the follow-up.

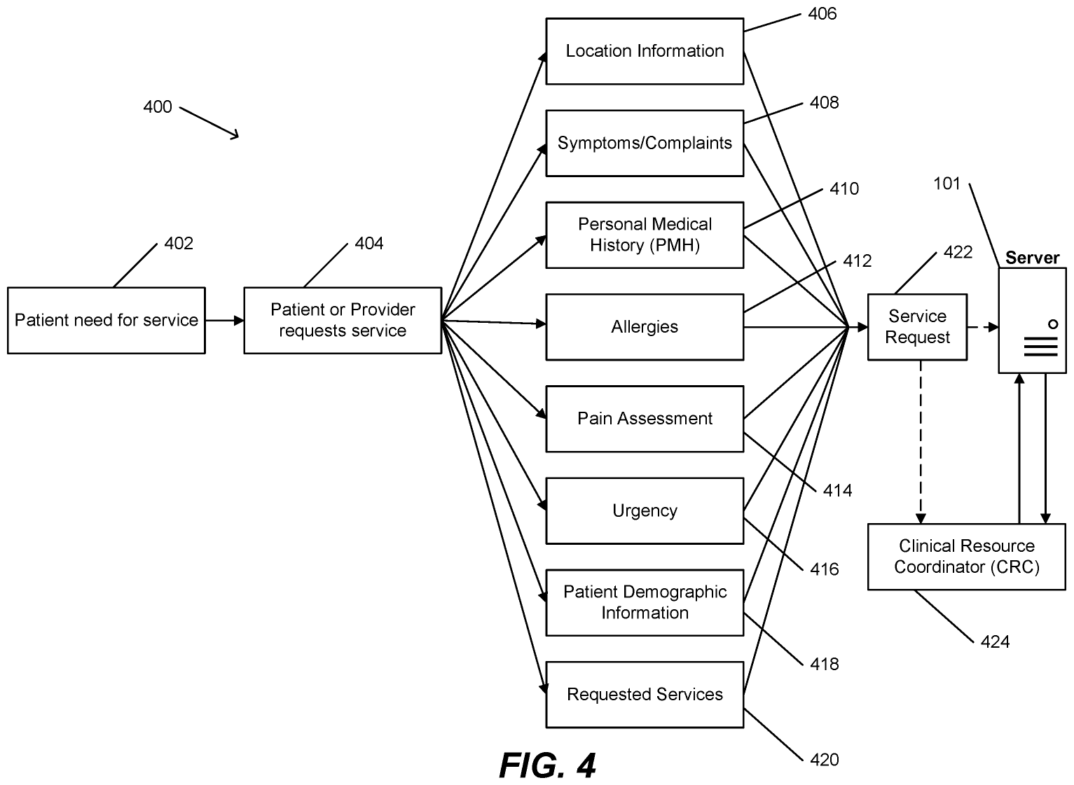

Referring to FIG. 4, a block flow diagram of a medical service request process 400 is shown. A first stage 402 of the process 400 involves the patient experiencing a need for medical service. At stage 404, the patient, a medical provider for the patient, or a caregiver for the patient, creates a request 422 for medical services. In general, the request 422 includes information associated with the patient and the current medical crisis, as well as historical and other information relevant to a medical diagnosis. In an example, the request 422 may include one or more of: location information of the patient 406; symptoms or complaints experienced by the patient 408; personal medical history (PMH) of the patient 410, which may include a history of present illness (HPI); allergies of the patient 412; a pain assessment submitted by the patient 414, which may be based on the pain Provocation/Palliation, Quality, Radiation/Region, Severity, Time of Onset (PQRST) scale and include a pain severity level between 0-10 and a written description of the pain's PQRST; an urgency determination 416, which may be a selection between discrete urgency levels such as “non-urgent”, “urgent”, and “serious”; patient demographic information 418, including information such as patient sex, gender, age, race, ethnicity, sexual orientation, or other medically relevant demographic information; and requested medical services 420. Other information may also be included in the service request 422. The service request 422 may be communicated directly to the server 101 via, for example, a web-based application with a user interface (UI), or it may be first communicated to a Clinical Resource Coordinator (CRC) 424 via, for example, a telephone call or a web-based instant message (IM) chat, and then communicated by the CRC 424 to the server 101. The CRC 424 may be a clinical triage nurse 106, and/or may be part of the clinical care team 102. The CRC 424 may also communicate with the sever 101 to amend, clarify, comment on, assess, and triage the service request 422.

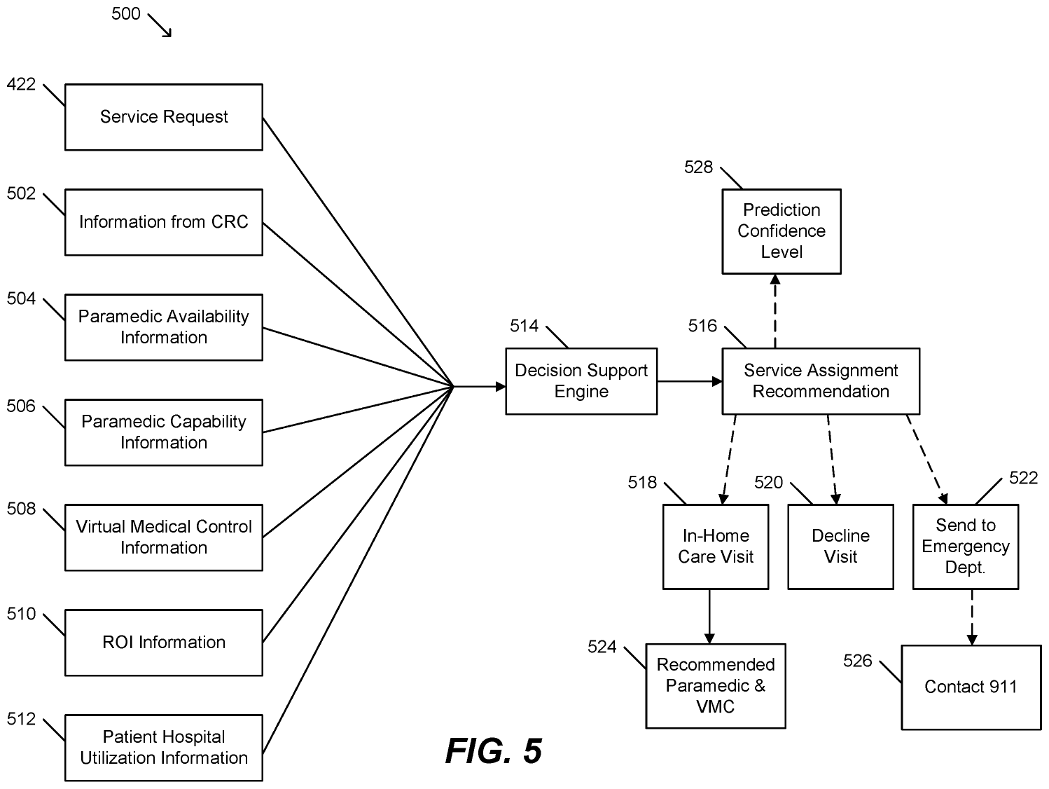

Referring to FIG. 5, with further reference to FIG. 4, a block flow diagram of an example of a decision support process 500 is shown. The server 101, including cloud computing configurations, is an example means for executing the process 500. In an example, a decision support engine 514 may be configured to receive one or more of a number of inputs such as: the service request 422, as described in FIG. 4; CRC information 502, which may include one or more of amendments to, clarifications of, comments on, assessments of, and triage of the service request 422; paramedic availability information 504, including, for example, the locations of paramedic, the service areas of the paramedics, the transportation means of the paramedics, the schedules of the paramedics, the current workloads of the paramedics, and the projected workloads of the paramedics; paramedic capability information 506, including, for example, experience, training, certifications, licensure, and other expertise of the paramedics, as well as medical tools and resources available to the paramedics; VMC information 508, including VMC availability information such as VMC activity, VMC workload, and VMC schedule, and VMC capability information, such as experience, training, certifications, licensure, and other expertise of the VMC(s); ROI information 510, including insurance assessment information, cost assessment information, ROI assessment information, and other information relating to the financial burdens and benefits of providing medical service to the patient; and patient hospital utilization information 512, which may include previous hospital visits by the patient, which may further include emergency department (ED) visits, which may even further include times when a CRC 424, a paramedic 112, a VMC 108, or the decision support engine 514 recommended that the patient visit the ED, including such times as when the decision support engine 514 did not recommend the patient visit the ED, but the CRC 424, paramedic 112, or VMC 108 referred the patient to the ED anyway.

The decision support engine 514 may be configured to process the inputs and generate a service assignment recommendation 516. The inputs may be received via several means including, e.g., a patient app, a provider app, and a paramedic app. The decision support engine 514 may process inputs by parsing them using natural language processing (NLP), including medical NLP (mNLP). The service assignment recommendation 516 may typically comprise a recommendation to perform an in-home care visit 518, a recommendation to decline an in-home care visit 520, or a recommendation to send the patient to the emergency department 522. The service assignment recommendation may be based, at least in part, on a determination of the level of risk posed by the patient's symptoms, the patient's PMH, or both. The recommendation 516 may include, and/or be based on the predicted number of medical services expected during the visit.

This determination may involve determining if the patient is experiencing a medical emergency, such as by parsing the service request 422 and determining if the patient is experiencing critical symptoms, is experiencing symptoms that, in combination, correspond to a critical illness, or has a critical illness in their medical history, or by determining if the sum of weighted risk values associated with the patient's symptoms exceeds a certain threshold. If a patient is experiencing a medical emergency, the decision support engine 514 may be configured to generate a recommendation to send the patient to the ED 522. The decision support engine may, concurrently with or subsequently to generating a recommendation to send the patient to the ED 522, contact emergency medical services on behalf of the patient. The decision support engine may be configured to provide instructions or other indications to the one or more users 102-112 to contact emergency medical services on behalf of the patient. For example, in the United States, the decision support engine 514 may be configured to contact 911 on behalf of the patient.

Whether illnesses and symptoms are critical, alone or in combination, may be determined by a machine learning (ML) model. In general, the ML model may be trained using diagnostic criteria data and other data. Its recommendations may be validated by decisions made by doctors or nurses in agreement or disagreement with the recommendations. The ML model may be trained periodically based on doctors and nurses agreeing or disagreeing with the recommendations made by the ML model. Whether illnesses and symptoms are critical, alone or in combination, may also be determined by manual input. In an example, the ML model utilizes supervised learning methods based on training data included in prior service requests 422 and inputs described in FIG. 5. Classification techniques may be used to predict output labels or responses for new input data based on what the ML model learned during the training phase. Regression techniques may also be used to train the ML model for numerical outputs such as the expected costs of a visit and other visit prediction information, such as a predicted count of services that will be provided during the visit. The output labels may include, for example, identifying critical symptoms and general symptoms based on the inputs, and determining a weight value for the general symptoms. The ML model may be configured to generate other output labels for new inputs based on the prior training.

The decision support engine 514 may be configured to utilize the ML model to determine if the patient is experiencing symptoms that, in combination, place the patient at serious risk, constituting a medical emergency. The decision support engine 514 may be configured to assign a weighted risk value to each symptom, and determine if the sum of the weighted risk values of the symptoms experienced by the patient exceeds a threshold value. In an example, the risk values may be determined or supplemented based on manual input by a user such as the VMC 108.

The decision support engine 514 may be configured to determine if medical resources are available to provide service to the patient. This may include determining whether there are one or more paramedics and VMCs available that are not preoccupied with other patients and/or do not have full schedules, whether these paramedics and VMCs have appropriate licensure, training, experience, and other expertise to treat the patient, whether the paramedic can reach the patient in an acceptable timeframe considering the patient's symptoms and/or conditions, and whether the cost of treating the patient will be too much as compared to the financial benefit.

If the decision support engine 514 determines that the patient is not experiencing a medical emergency and that there are medical resources available to provide service to the patient, it may generate a recommendation to provide an in-home care visit 518. The decision support engine may, concurrently or subsequently with generating a recommendation to provide an in-home care visit 518, generate a recommendation 524 to choose a particular paramedic to perform the in-home care visit and a particular VMC to supervise the visit. This recommendation 524 may be based, at least in part, on paramedic availability information 504, paramedic capability information 506, and VMC information 508.

If the decision support engine 514 determines that the patient is not experiencing a medical emergency, but that there are not sufficient resources available to provide service to the patient, it may generate a recommendation to decline an in-home care visit. The decision support engine 514 may also recommend to decline a visit 520 if the requested and/or required medical services/interventions are outside the services offered by the paramedics/VMCs. This recommendation may be based, at least in part, on paramedic availability information 504, paramedic capability information 506, and VMC information 508.

When the decision support engine 514 generates a recommendation 518-522, it may display such recommendation to one or more users 102-112, including preferably to the CRC 424. It may additionally display a prediction confidence level 528, which indicates a score associated with the level of confidence that the system has determined corresponds to the recommendation. The prediction confidence level 528 may be displayed as a percentage. The prediction confidence level 528 may further include information of the factors that most affect the score. For example, if the decision support engine 514 generates a recommendation 522 to send the patient to the ED, it may generate and display a prediction confidence score of 80% as well as an indication that the greatest factor affecting the score is the patient's history of cancer. The prediction confidence level 528 may be calculated using an algorithm such as, e.g., the XBoost algorithm, a custom algorithm, or another algorithm known in the art.

The decision support engine 514 may require different levels of review of the recommendation 516, depending on the prediction confidence level 528. For example, if the prediction confidence level 528 is below a threshold level of confidence, then it may require review by one or more CRCs 424 (which may be clinical triage nurse(s) 106) and/or VMCs 108. If the prediction confidence level 528 is above a threshold level of confidence, it may require review by only one CRC 424. In another example, if the CRC 424 disagrees with the recommendation 516, it may trigger an additional review by another CRC 424 or a VMC 108.

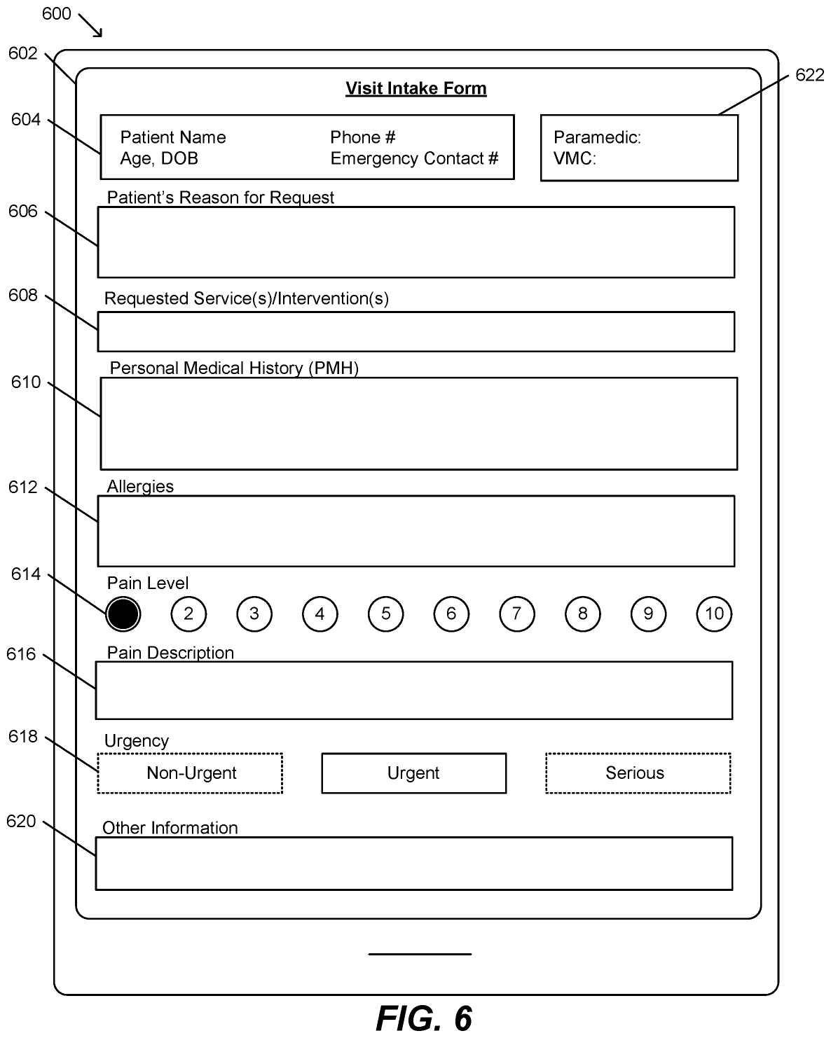

Referring to FIG. 6, an example user interface of a medical service visit request intake form user interface (UI) 600 is shown. The visit intake form user interface UI 600 is exemplary only and other UIs may be used to receive input of patient or provider 104 request information. The visit intake form UI 600 is displayed on a screen 602. The screen 602 may be a screen of any electronic device, including a computer, smartphone, or tablet. The visit intake form UI 600 allows a user, such as the patient's caregiver 104, to interact with the server in order to submit a medical service request 422. The layout, data fields and UI objects described on the UI 600 are examples and not limitations. Other layouts, data fields, and associated graphical UI objects such as radio buttons, command buttons, lists boxes, combination boxes, selectors, sliders, etc. as known in the art may be utilized in the UI 600.

The visit intake form UI 600 may comprise one or more of the following input fields: a patient information field 604, where a user may input the patient's name, age, date of birth (DOB), phone number, and emergency contact number; a reason for request field 606, where a user may input the symptoms, conditions, and circumstances leading to the request for medical services; a requested services field 608, where a user may input specific medical services or interventions sought to be provided; a personal medical history field 610, where a user may input relevant PMH information of the patient, including, e.g., current illnesses, past illnesses, HPI, chronic conditions, past diagnoses, and past surgeries; an allergies field 612, where a user may input relevant allergies known to be experienced by the patient including, e.g., allergies to latex or penicillin; a pain level field 614, where a user may input the severity of pain currently experienced by the patient, which may include selecting a number between 1 and 10 to indicate pain severity; a pain description field 616, where a user may input a description of the pain experienced by the patient, including, e.g., PQRT information; an urgency field 618, where a user may input the urgency of the patient's need for medical services, and which may comprise discrete selectable options such as “non-urgent”, “urgent”, and “serious”; and an other information field 620, where a user may input other relevant information relating to the patient's request for medical services. The visit intake form UI 600 may also comprise a field 622 displaying a paramedic and VMC if such medical professionals have been assigned to address the patient's request.

The visit intake form UI 600 may comprise other fields and other information in addition to those shown in FIG. 6. For example, it may comprise a field for affected body systems, which may be filled by a user selecting one or more discrete selectable options of affected body systems. In a further example, the server 101 may parse the request 422 to determine, based on keywords, affected body systems and display, on the visit intake form UI 600, recommended body systems to select as being currently affected. The visit intake form UI 600 may also comprise questions directed to the patient, the answers to which will provide important information to the medical professionals such as the CRC 424, VMC 108, and paramedic 112. The visit intake form UI 600 may also receive a requestor's goals and expected outcomes, and requested services/interventions such as when a referring provider submits the request on behalf of the patient.

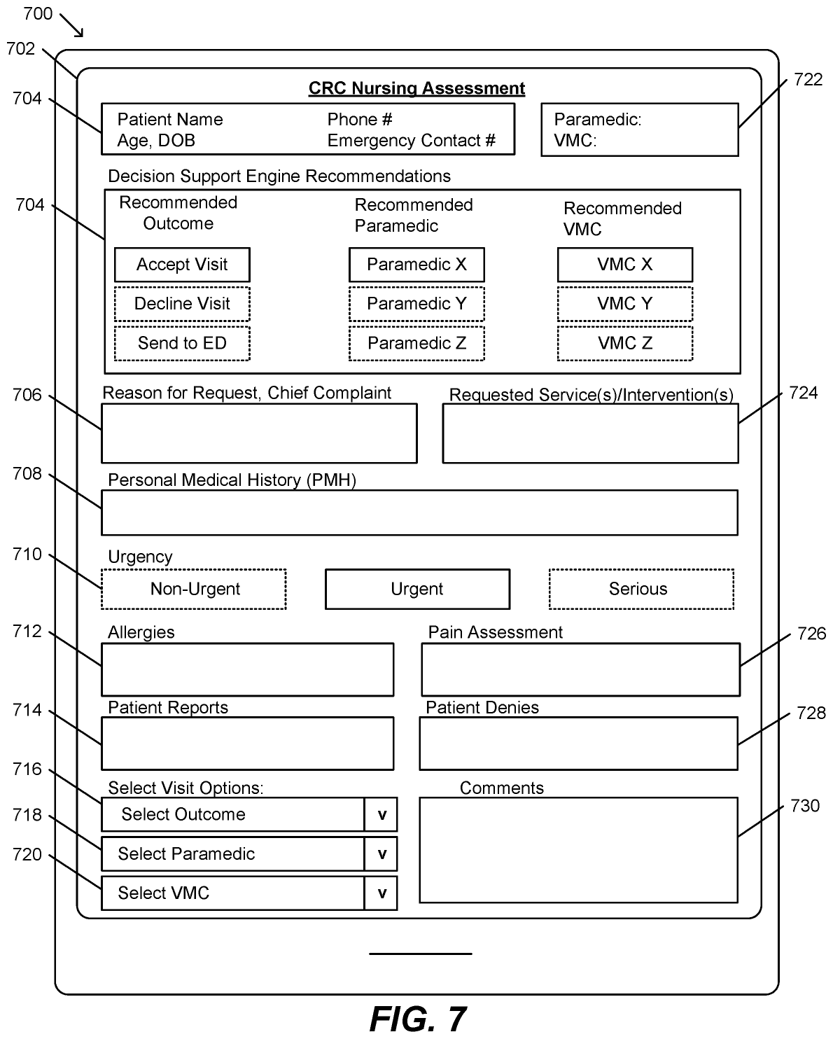

Referring to FIG. 7, an example user interface of a medical service visit request assessment form is shown. The visit request assessment form UI 700 is also exemplary only and other layouts, data fields and UI objects may be used. The visit request assessment form UI 700 is displayed on a screen 702. The screen 702 may be a screen of any electronic device, including a computer, smartphone, or tablet. The visit request assessment form UI 700 may allow a user, such as a CRC 424, interact with the server 101 in order to amend, clarify, comment on, assess, and triage the service request 422.

The visit request assessment form UI 700 may comprise one or more of the following editable display fields: a patient information field 704, which may display a patient's name, age, DOB, phone number, and emergency contact number; a decision support engine recommendations field 704, which may display, at least, recommended options generated by the decision support engine 514, such as a recommended outcome of the request and, if relevant, a recommended paramedic and a recommended VMC; a reason for request field 706, which may display the patient's symptoms, conditions, and circumstances leading to the request for medical services; a requested services field 724, which may display specific medical services or interventions sought to be provided; a personal medical history field 708, which may display relevant PMH information of the patient, including, e.g., current illnesses, past illnesses, HPI, chronic conditions, past diagnoses, and past surgeries; an urgency field 710, which may display the urgency of the patient's need for medical services, and which may comprise discrete selectable options such as “non-urgent”, “urgent”, and “serious”; an allergies field 712, which may display relevant allergies known to be experienced by the patient including, e.g., allergies to latex or penicillin; a pain assessment field 726, which may display the severity and description of the pain currently experienced by the patient; a patient reports field 714, which may display, e.g., body systems selected as being affected and questions about the patient's condition that were answered in the affirmative; a patient denies field 728, which may display, e.g., body systems suggested but not selected as being affected and questions about the patient's condition that were answered in the negative; visit options selection fields 716, 718, & 720, which allow the user, such as the CRC 424, to select, for example, a visit request outcome, a selected paramedic, and a selected VMC, respectively; and a comments field 730, where a user, such as the CRC 424, may provide comments on the patient's service request 422, including, e.g., comments about possible diagnoses or recommended interventions. The visit request assessment form UI 700 may also comprise a field 722 displaying a paramedic and VMC if such medical professionals have been assigned to address the patient's request.

The visit request assessment form UI 700 may comprise other fields and other information not shown in FIG. 7. For example, it may display a prediction confidence level 528 adjacent to the decision support engine recommendations field 704. In another example, the UI 700 may provide alerts, such as when the volume of requests reaches a critical number, when the inventory of medical tools or resources reaches a critical number, when the available medical care providers (including paramedics and VMCs) reach a critical schedule capacity, when a patient request has not been triaged or assigned after a certain period of time, or when a patient has not been treated after a certain period of time.

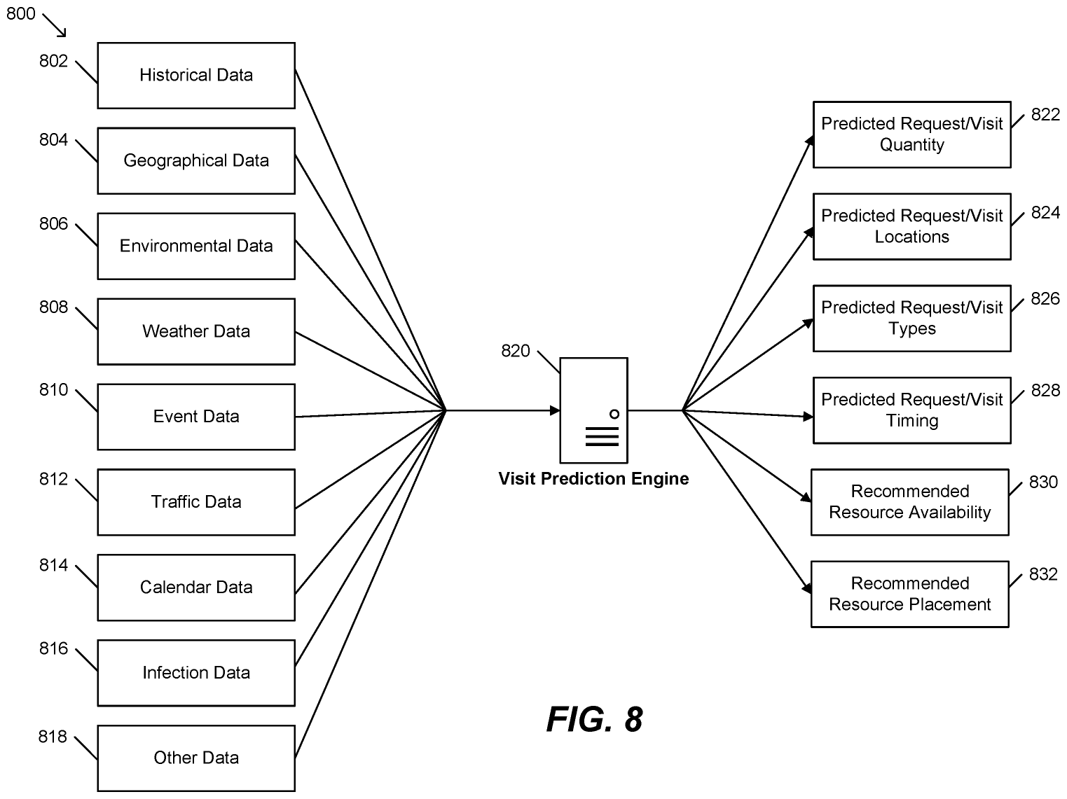

Referring to FIG. 8, a block flow diagram of a visit prediction process 800 is shown. The server 101, including cloud computing configurations, is an example means for executing the process 800. In general, a visit prediction engine (VPE) 820 may be configured to receive one or more of a number of inputs and provide one or more outputs to facilitate resource management for the medical aid dispatching techniques described herein. The inputs and outputs included in the process 800 are examples, and not limitations as other inputs may be used to train a ML module within the VPE 820 and produce the predictive outputs.

The one or more inputs to the VPE 820 may include historical data 802, which may comprise data of previous dates, visit requests, and visits, including, for example, quantity, timing, location, reasons for request, selected outcome, time between request and service, time spent en route, medical resource availability, medical resource placement, and medical resource utilization.

Inputs may include geographical data 804, which may comprise data of a particular region, including, for example, a geographical map of roads, population density (including time-specific population density), address locations, hospital locations, urgent care locations, road closures, and topography. Inputs may include environmental data 806, which may comprise data of a local environment including, for example, air quality, water quality, rain acidity, smog, smoke, bacteria, and algae. Weather data 808, which may comprise data of a particular region, including, for example, a forecast, weather patterns, temperature, wind speed and direction, precipitation levels, snow and slush accumulation, ice hazards, hail, flash floods, lightning storms, tornados, earthquakes, visibility, and other weather effects may be input to the VPE 820. Event data 810, which may include, for example, information of past, current, or upcoming events, such as concerts, conventions, speeches, sporting events, or other gatherings of people, and traffic data 812, which may include, for example, traffic congestion, traffic speed, traffic duration, traffic patterns, route length, route duration, and speed limits may be utilized by the VPE 820. Temporal based data such as calendar data 814, which may include, for example, time of day, dates, weekdays, weekends, months, holidays, daylight savings switch days, and other days of importance may be inputs to the VPE 820. Inputs may include infection data 816, which may include, for example, infection rates of various communicable diseases, viruses, and bacterial infections such as, e.g., COVID-19 and influenza. Other data 818, which may comprise any relevant data not previously mentioned, may also be utilized by the VPE 820. All of the aforementioned input data may be past data, present data, or anticipated future data.

The VPE 820 may, based on one or more of these inputs, generate one or more outputs. An output may include a predicted request/visit quantity 822, which may comprise a prediction of how may visit requests to expect on a particular day or other period of time, and, optionally, a prediction of how many of these visit requests to expect to proceed into home care visits. The output may include predicted request/visit locations 824, which may include predictions of the general or specific locations of expected visit requests and subsequent visits, and predicted request/visit types 826, which may include predictions of the reasons for expected visit requests and subsequent medical services or interventions provided. The output may include predicted request/visit timing 828, which may include predictions of the times and dates of expected visit requests and subsequent visits, and recommended resource availability 830, which may recommend, e.g., a quantity of paramedics 112, VMCs 108, CRCs 424, paramedic dispatchers 110, trucks, ambulances, and medical tools. The output may include recommended resource placement 832, which may recommend, e.g., initial placement of medical resources (including paramedics 112) to align with the predicted request/visit quantity 822 and locations 824. The VPE 820 may be configured to provide additional outputs based on the training data.

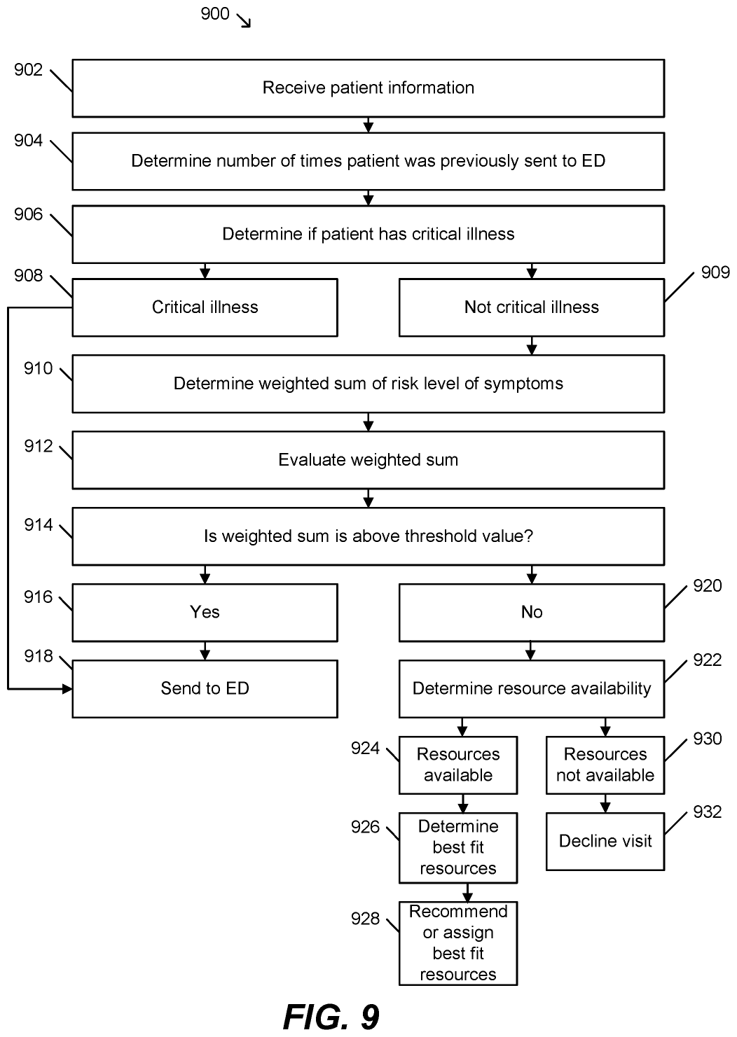

Referring to FIG. 9, a block flow diagram of an embodiment of an example request-outcome decision support algorithm 900 is shown. The server 101, including cloud computing configurations, is an example means for executing the algorithm 900. The algorithm 900 is, however, an example only and not limiting. The algorithm 900 may be altered, e.g., by having stages added, removed, rearranged, combined, performed concurrently, and/or having single stages split into multiple stages.

At stage 902, the algorithm includes receiving patient information. This may typically be in the form of a service request 422. At stage 904, it is determined how many times the patient has previously been sent to the ED. At stage 906, the algorithm includes determining if the patient has a critical illness. This determination may be made with the assistance of an ML model. If the patient is determined to have a critical illness at stage 908, then the patient may be sent to the ED at stage 918. If the patient is not determined to have a critical illness at stage 909, then at stage 910 the algorithm includes determining a weighted sum of the risk levels of the symptoms experienced by the patient. The weighted sum may be determined with the assistance of an ML model. The ML model may be initially trained using initial risk values set by medical professionals. Each symptom may be assigned a risk value from 1-5. As an example, abdominal pain may have a risk score of 2, while nausea/vomiting has a risk score of 3, while shortness of breath has a risk score of 4. The ML model may then be trained subsequently, via supervised learning, to update the risk scores of each symptom based on the frequency that symptom appears in patients who are sent to the ED. For example, if patients experiencing abdominal pain have been sent to the ED at a higher rate than expected based on the risk score, then the ML model may adjust the risk score of abdominal pain upwards (e.g., to 3).

At stage 912, the algorithm includes evaluating the weighted sum. In an example, the evaluation of the weighted sum may be based on the output of an ML model. The ML model may, for example, adjust the risk value of one or more patient symptoms, or of the weighted sum of patient symptoms, based on certain patient information, such as, e.g., patient age, patient sex, and patient PMH. For example, if the patient is of an advanced age, the ML model may increase the weighted sum by a particular factor. In another example, if the patient has a history of a particular disease in their PMH, then the ML model may adjust the risk value of particular symptoms that correspond to that disease based on a particular factor. The ML model may be trained initially based on initial inputs provided by medical professionals. The ML model may be trained subsequently, via supervised learning based on visit outcome data, to adjust risk factors associated with patient demographic information and patient PMH.

At stage 914, the algorithm may include comparing the weighted sum of the risk levels to a threshold value. The threshold value may vary based on the patient information and other related patient history. For example, the threshold value may be based at least in part on the number of times the patient has been sent to the ED, determined at stage 904 (e.g., the threshold value of risk may be lower if the patient has been sent to the ED before). If the weighted sum is above the threshold value of risk, as at stage 916, then the algorithm may determine to send the patient to the ED at stage 918. If the weighted sum is not above the threshold value of risk, as at stage 920, then medical resource availability may be determined at stage 922.

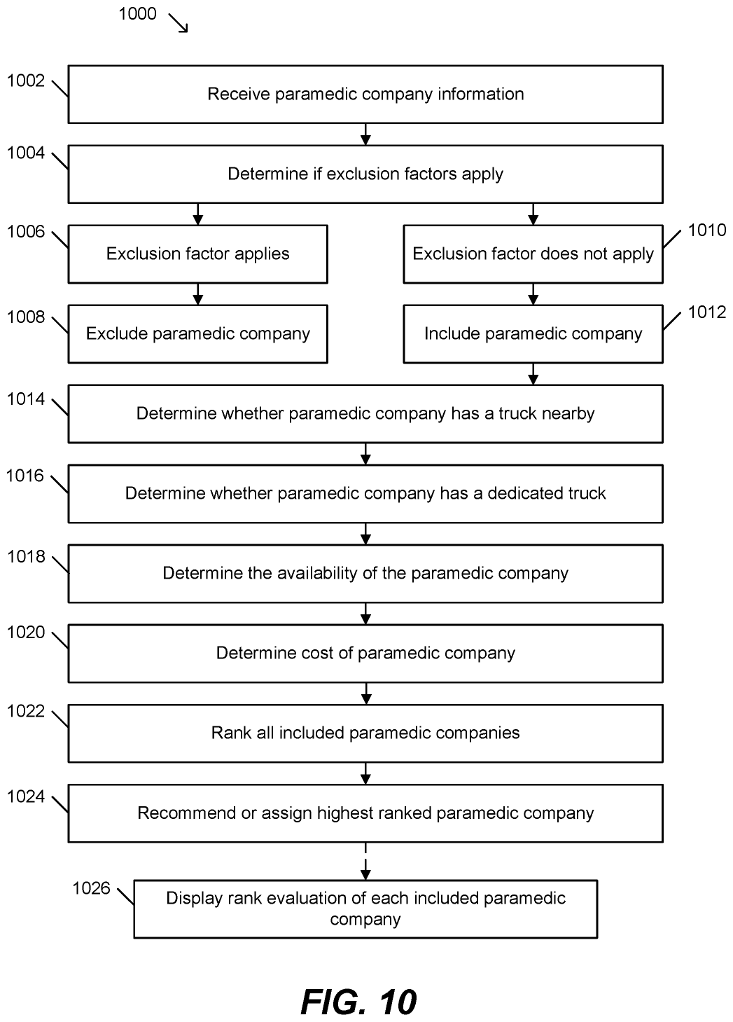

If sufficient medical resources are available, as at stage 924, then the algorithm may, at stage 926, determine the best fit resources for the patient. This determination may involve consideration of, e.g., the proximity of the available paramedics, the competency of the available paramedics and VMCs, the medical tools required of the patient and the medical tools available and proximate, the urgency of the request, the time it will take to reach the patient, the likelihood of successful intervention, the cost of the visit, the financial benefits of the visit, and any other relevant factors. This determination may also involve use of an algorithm such as the embodiment shown in FIG. 10. This determination may also be made with the assistance of an ML model. At stage 928, the algorithm may recommend the determined best-fit medical resources. This recommendation may be made to a user, including a CRC 424, via a web-application-based UI, such as UI 700. At stage 928, the algorithm may, optionally, instruct the server 101 to assign the determined best-fit resources to the patient. If sufficient medical resources are not available, as at stage 930, then the algorithm may recommend a user, including a CRC 424, to decline a visit, or, optionally, instruct the server 101 to decline the visit.

Referring to FIG. 10, a block flow diagram of an embodiment of an example medical resource selection algorithm 1000 is shown. The medical resource selection algorithm may be utilized in the decision support algorithm 900 at, e.g., stages 922-926. The server 101, including cloud computing configurations, is an example means for executing the algorithm 1000. The algorithm 1000 is, however, an example only and not limiting. The algorithm 1000 may be altered, e.g., by having stages added, removed, rearranged, combined, performed concurrently, and/or having single stages split into multiple stages.

At stage 1002, the algorithm includes receiving paramedic company information. Paramedic company information may include, for example, geographic regions served by the paramedic companies, the number of available paramedics in each company, the competencies of the paramedics in each company, the medical tools available to each company, the number and location of trucks owned by (or available to) each paramedic company, the cost of utilizing the trucks of each paramedic company, and whether the trucks owned by (or available to) each paramedic company are dedicated for in-home visits processed by the system 100 (i.e. a dedicated truck).

At stage 1004, the algorithm may determine if at least one exclusion factor applies to any of the paramedic companies. Exclusion factors may include, e.g., if the paramedic company does not provide service in the region of the patient. If an exclusion factor does apply to a paramedic company, as at stage 1006, then that paramedic company may be excluded at stage 1008. If an exclusion factor does not apply to a paramedic company, as at stage 1010, then that paramedic company will be included in further determinations, as at stage 1012. At stage 1014, the algorithm may determine whether each included paramedic company has an available truck nearby. At stage 1016, the algorithm may determine whether each included paramedic company has a dedicated truck. At stage 1018, the algorithm may determine the level of availability of each included paramedic company. The level of availability may be categorized in discrete categories such as, e.g., “available now”, “available soon”, “available later” and “not available”. At stage 1020, the algorithm may determine the estimated cost of employing each included paramedic company for the visit to the patient. The determinations at stages 1014-1020 may involve assigning weighted values to each included paramedic company based on the determinations.

At stage 1022, the algorithm includes ranking the paramedic companies. The ranking may take into account the information received at stage 1002, the determinations made at stages 1014-1020, and other relevant information. The ranking may include ranking the summed weighted values assigned to each paramedic company. At stage 1024, the algorithm may recommend the highest ranked paramedic company to be assigned to the patient. This recommendation may be displayed to a user, including a CRC 424, via a web-application-based UI, such as UI 700. Optionally, at stage 1024, the algorithm may instruct server 101 to assign the highest ranked paramedic company to the patient. At stage 1026, the rank evaluation of each included paramedic company may be displayed to a user, such as a CRC 424.

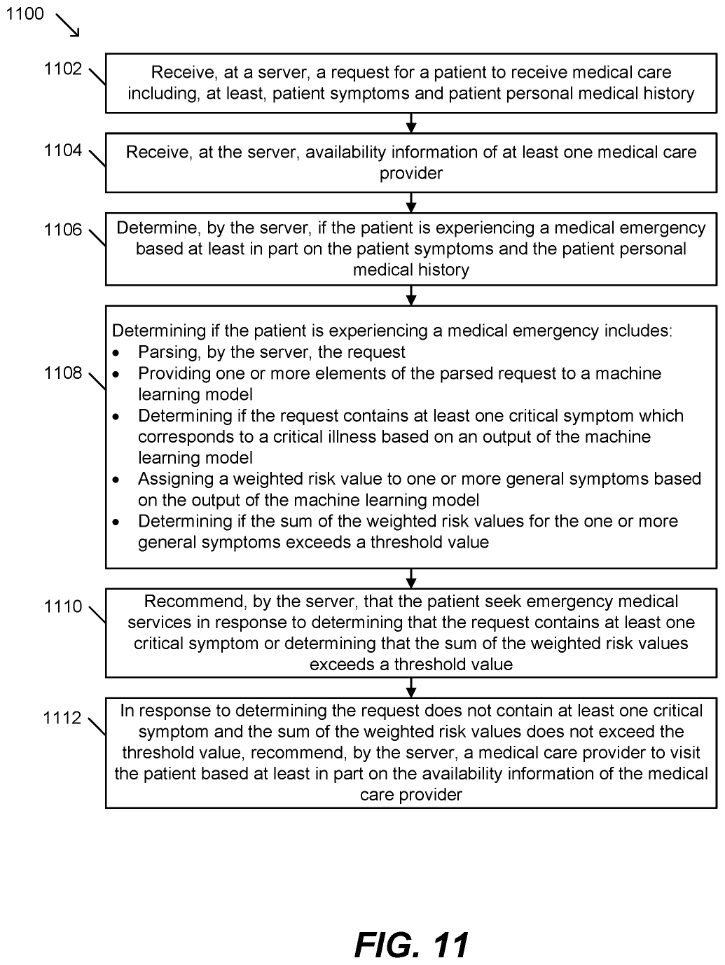

Referring to FIG. 11, a block flow diagram of a method of assisting with medical care dispatch 1100 is shown. This method 1100 may be carried out by a server 101, including the cloud-based configurations of the server 101. The method 1100 is, however, an example only and not limiting. The method 1100 may be altered, e.g., by having stages added, removed, rearranged, combined, performed concurrently, and/or having single stages split into multiple stages.

At stage 1102, method includes receiving, at a server, a request for a patient to receive medical care including, at least, patient symptoms and patient personal medical history. In an example, a patient, medical provider, or caregiver 104 may utilize an application (e.g., via a smartphone) or other web/browser based application to provide the patient symptoms and medical history. The CRC 424 may input the patient information to the server. A GUI, such as depicted in FIG. 6, is an example for receiving the request for medical care. The patient information may also include one or more of location information, allergies, pain assessment (including severity and description), urgency, patient demographic information, requested medical services, and insurance information.

At stage 1104, the method includes receiving, at the server, availability information of at least one medical care provider. The availability information may include information relating to the service areas of the medical care provider (e.g. a paramedic and a VMC), information relating to the proximity of the truck (including, e.g., an ambulance) of the at least one medical provider in relation to the patient, information of whether the at least one medical care provider has a dedicated truck, information of the at least one medical care provider's schedule, information of the at least one medical provider's competency and licensure, information of the route between the at least one medical provider and the patient, information of the traffic on the route between the at least one medical provider and the patient, and information relating to the cost and/or financial benefit of providing service to the patient.

At stage 1106, the method includes determining, by the server, if the patient is experiencing a medical emergency based at least in part on the patient symptoms and the patient personal medical history. A medical emergency may be determined based on the patient experiencing a critical illness, critical symptoms, or symptoms that, in combination, place the patient in significant risk of a serious adverse health outcome. Critical illnesses, critical symptoms, and risk levels of symptoms may be determined by medical professionals, and/or they may be determined with the assistance of an ML model. The ML model may be trained using data of symptoms and illnesses associated with health outcomes, including, e.g., trips to the ED, permanent disability, and death.

At stage 1108, the determining if the patient is experiencing a medical emergency includes: parsing, by the server, the request; providing one or more elements of the parsed request to a machine learning model; determining if the request contains at least one critical symptom which corresponds to a critical illness based on an output of the machine learning model; assigning a weighted risk value to one or more general symptoms based on the output of the machine learning model; determining if the sum of the weighted risk values for the one or more general symptoms exceeds a threshold value.

The parsing by the server and the ML model may include using natural language processing (NLP) including medical natural language processing (mNLP). The determining if the request contains at least one critical symptom which corresponds to a critical illness may include evaluating associations between symptoms and critical illnesses. These associations may be learned by the ML model or input to the ML model. The assigning a weighted risk value to one or more general symptoms based on the output of the ML model may include evaluating associations between symptoms and risk of serious adverse health outcomes. These associations may be learned by the ML model based on data including patient symptoms, medical interventions, and patient health outcomes.

Before assigning weighted risk values, the ML model may be trained using initial risk values set by medical professionals. Each symptom may be assigned a risk value from 1-5. As an example, abdominal pain may have a risk score of 2, while nausea/vomiting has a risk score of 3, while shortness of breath has a risk score of 4. Other symptoms may be associated with similar risk scores. The ML model may then be trained subsequently, via supervised learning, to update the risk scores of each symptom based on the frequency that symptom appears in patients who are sent to the ED. For example, if patients experiencing abdominal pain have been sent to the ED at a higher rate than expected based on the risk score, then the ML model may adjust the risk score of abdominal pain upwards (e.g., to 3).

Before determining if the sum of the weighted risk values exceeds a threshold value, the ML model may, for example, adjust the risk value of one or more patient symptoms, or of the weighted sum of patient symptoms, based on certain patient information, such as, e.g., patient age, patient sex, and patient PMH. For example, if the patient is of an advanced age, the ML model may increase the weighted sum by a particular factor. If the patient has a history of a particular disease in their PMH, then the ML model may adjust the risk value of particular symptoms that correspond to that disease based on a particular factor. The ML model may be trained initially based on initial inputs provided by medical professionals. The ML model may be trained subsequently, via supervised learning based on visit outcome data, to adjust risk factors associated with patient demographic information and patient PMH.

At stage 1110, the method includes recommending, by the server, that the patient seek emergency medical services in response to determining that the request contains at least one critical symptom or determining that the sum of the weighted risk values exceeds a threshold value. The recommending may be made to one or more users, including the patient's caregiver 104, the clinical triage nurse 106, the CRC 424, the VMC 108, and the paramedic 112. The recommending may be made via a GUI such as the one disclosed in FIG. 7. In some embodiments, the server may contact emergency medical services (such as 911) on behalf of the patient.

At stage 1112, the method includes recommending, by the server, in response to determining the request does not contain at least one critical symptom and the sum of the weighted risk values does not exceed the threshold value, a medical care provider to visit the patient based at least in part on the availability information of the medical care provider. The medical care provider may include a paramedic 112. The recommending may be made to one or more users, including the clinical triage nurse 106, the CRC 424, the VMC 108, the paramedic dispatcher 110, and the paramedic 112. The recommending may be made via a GUI such as the one disclosed in FIG. 7. The server may assign the recommended medical care provider to the patient, for the purposes of providing an in-home visit in response to the patient's medical service request 422.

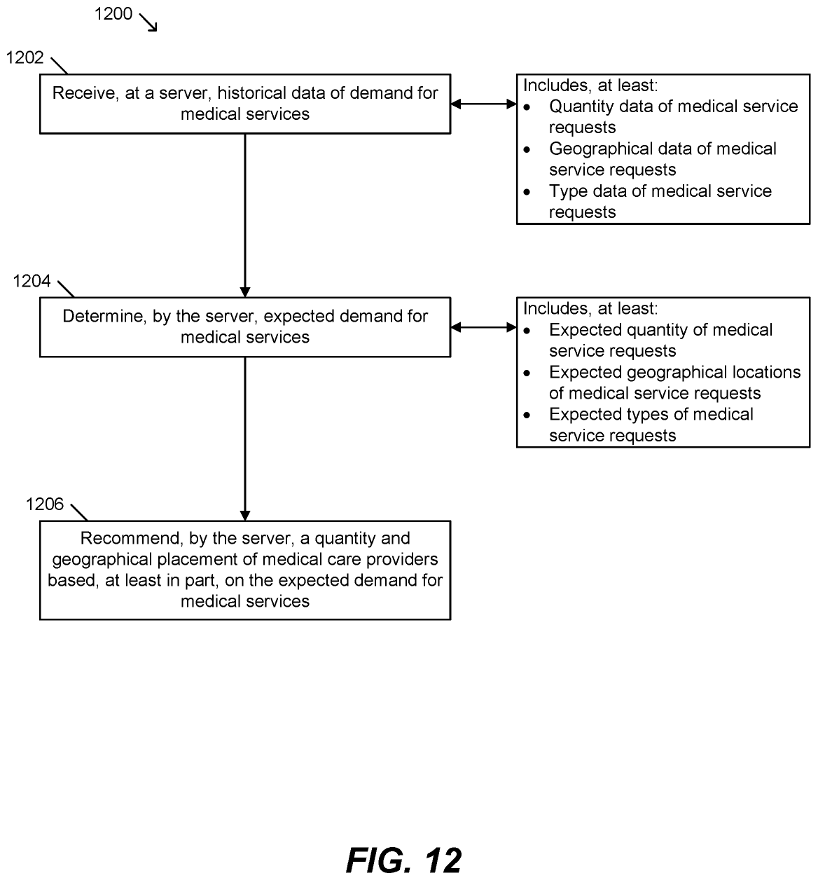

Referring to FIG. 12, a block flow diagram of a method of anticipating demand for medical services 1200 is shown. This method 1200 may be carried out by a server 101, including the cloud-based configurations of the server 101. The method 1200 is, however, an example only and not limiting. The method 1200 may be altered, e.g., by having stages added, removed, rearranged, combined, performed concurrently, and/or having single stages split into multiple stages.

At stage 1202, the method includes receiving, at a server, historical data of demand for medical services. This historical data may include, at least, quantity data of medical service requests, geographical data of medical service requests, and type data of medical service requests. The historical data may be historical data 802, which may comprise data of previous dates, visit requests, and visits, including, for example, quantity, timing, location, reasons for request, selected outcome, time between request and service, time spent en route, medical resource availability, medical resource placement, and medical resource utilization.

At stage 1204, the method includes determining, by the server, expected demand for medical services. This expected demand may include, at least, expected quantity of medical service requests, expected geographical locations of medical service requests, and expected types of medical service requests. This determining may be performed by an ML model. The ML model may, for example, determine trends and associations from the historical data, including of medical service request quantity, geographical location, type, timing, reasons, outcome, time between request and service, time spent en route, medical resource availability, medical resource placement, and medical resource placement, in relation to various factors. The factors may be identified from various inputs including: geographical data, environmental data, weather data, event data, traffic data, calendar data, infection data, and other data. The ML model may receive supervised or unsupervised training based on new data (e.g. historical data) that accumulates, and it may establish, based on this training, new or different trends and associations.

At stage 1206, the method includes recommending, by the server, a quantity and geographical placement of medical care providers based, at least in part, on the expected demand for medical services. The recommending may be to one or more users, including the clinical care team 102, the clinical triage nurse 106, the virtual medical control 108, the paramedic dispatcher 110, the paramedic 112, the CRC 424, or other individuals. The recommendation may be displayed on a GUI.

Other examples and implementations are within the scope and spirit of the disclosure and appended claims. For example, due to the nature of software and computers, functions described above can be implemented using software executed by a processor, hardware, firmware, hardwiring, or a combination of any of these. Features implementing functions may also be physically located at various positions, including being distributed such that portions of functions are implemented at different physical locations.

Also, as used herein, “or” as used in a list of items prefaced by “at least one of” or prefaced by “one or more of” indicates a disjunctive list such that, for example, a list of “at least one of A, B, or C,” or a list of “one or more of A, B, or C,” or “A, B, or C, or a combination thereof” means A or B or C or AB or AC or BC or ABC (i.e., A and B and C), or combinations with more than one feature (e.g., AA, AAB, ABBC, etc.).

As used herein, unless otherwise stated, a statement that a function or operation is “based on” an item or condition means that the function or operation is based on the stated item or condition and may be based on one or more items and/or conditions in addition to the stated item or condition.

Further, an indication that information is sent or transmitted, or a statement of sending or transmitting information, “to” an entity does not require completion of the communication. Such indications or statements include situations where the information is conveyed from a sending entity but does not reach an intended recipient of the information. The intended recipient, even if not actually receiving the information, may still be referred to as a receiving entity, e.g., a receiving execution environment. Further, an entity that is configured to send or transmit information “to” an intended recipient is not required to be configured to complete the delivery of the information to the intended recipient. For example, the entity may provide the information, with an indication of the intended recipient, to another entity that is capable of forwarding the information along with an indication of the intended recipient.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

The terms “machine-readable medium” and “computer-readable medium,” as used herein, refer to any medium that participates in providing data that causes a machine to operate in a specific fashion. Using a computer system, various computer-readable media might be involved in providing instructions/code to processor(s) for execution and/or might be used to store and/or carry such instructions/code (e.g., as signals). In many implementations, a computer-readable medium is a physical and/or tangible storage medium. Such a medium may take many forms, including but not limited to, non-volatile media and volatile media. Non-volatile media include, for example, optical and/or magnetic disks. Volatile media include, without limitation, dynamic memory.

Common forms of physical and/or tangible computer-readable media include, for example, a floppy disk, a flexible disk, hard disk, magnetic tape, or any other magnetic medium, a CD-ROM, any other optical medium, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to one or more processors for execution. Merely by way of example, the instructions may initially be carried on a magnetic disk and/or optical disc of a remote computer. A remote computer might load the instructions into its dynamic memory and send the instructions as signals over a transmission medium to be received and/or executed by a computer system.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, some operations may be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory computer-readable medium such as a storage medium. Processors may perform one or more of the described tasks.

Components, functional or otherwise, shown in the figures and/or discussed herein as being connected, coupled (e.g., communicatively coupled), or communicating with each other are operably coupled. That is, they may be directly or indirectly, wired and/or wirelessly, connected to enable signal transmission between them.

Having described several example configurations, various modifications, alternative constructions, and equivalents may be used without departing from the spirit of the disclosure. For example, the above elements may be components of a larger system, wherein other rules may take precedence over or otherwise modify the application of the disclosure. Also, a number of operations may be undertaken before, during, or after the above elements are considered. Accordingly, the above description does not bound the scope of the claims.

“About” and/or “approximately” as used herein when referring to a measurable value such as an amount, a temporal duration, and the like, encompasses variations of ±20% or ±10%, ±5%, or +0.1% from the specified value, as appropriate in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or +0.1% from the specified value, as appropriate in the context of the systems, devices, circuits, methods, and other implementations described herein.

A statement that a value exceeds (or is more than or above) a first threshold value is equivalent to a statement that the value meets or exceeds a second threshold value that is slightly greater than the first threshold value, e.g., the second threshold value being one value higher than the first threshold value in the resolution of a computing system. A statement that a value is less than (or is within or below) a first threshold value is equivalent to a statement that the value is less than or equal to a second threshold value that is slightly lower than the first threshold value, e.g., the second threshold value being one value lower than the first threshold value in the resolution of a computing system.

Further, more than one invention may be disclosed.

Implementation examples are described in the following numbered clauses:

Clause 1. A method for assisting with medical care dispatch, comprising: receiving, at a server, a request for a patient to receive medical care including, at least, patient symptoms and patient personal medical history; receiving, at the server, availability information of at least one medical care provider; determining, by the server, if the patient is experiencing a medical emergency based at least in part on the patient symptoms and the patient personal medical history; wherein the determining if the patient is experiencing the medical emergency includes parsing, by the server, the request, providing one or more elements of the parsed request to a machine learning model, determining if the request contains at least one critical symptom which corresponds to a critical illness based on an output of the machine learning model, assigning a weighted risk value to one or more general symptoms based on the output of the machine learning model, and determining if a sum of the weighted risk values for the one or more general symptoms exceeds a threshold value; recommending, by the server, that the patient seek emergency medical services in response to determining that the request contains at least one critical symptom or determining that the sum of the weighted risk values exceeds the threshold value; and in response to determining the request does not contain at least one critical symptom and the sum of the weighted risk values does not exceed the threshold value, recommending, by the server, a medical care provider to visit the patient based at least in part on the availability information of the medical care provider.

Clause 2. The method of clause 1, further comprising determining, by the server, a confidence score associated with a recommendation that the medical care provider visit the patient.

Clause 3. The method of clause 1, further comprising receiving, by the server, expertise information of at least one medical care provider.

Clause 4. The method of clause 3, wherein the recommending, by the server, the medical care provider is based at least in part on the expertise information of the medical care provider.

Clause 5. The method of clause 1, wherein the availability information also includes cost information.

Clause 6. The method of clause 1, wherein the recommending, by the server, that the patient seek emergency medical service further comprises contacting the emergency medical services.

Clause 7. The method of clause 1, wherein the request is received via a patient portal executing in a web browser.

Clause 8. The method of clause 1, wherein the availability information is received via a provider portal executing in a web browser.

Clause 9. The method of clause 1 wherein the one or more elements of the parsed request includes at least one of a location, a symptom, a medical history information, an indication of allergies, an indication of pain, and demographic information.

Clause 10. A system for assisting with medical care dispatch comprising: at least one server including a memory and at least one processor communicatively coupled to the memory and configured to: receive a request for a patient to receive medical care including, at least, patient symptoms and patient personal medical history; receive availability information of at least one medical care provider; determine if the patient is experiencing a medical emergency based at least in part on the patient symptoms and the patient personal medical history; wherein the determining if the patient is experiencing the medical emergency includes parsing the request, providing one or more elements of the parsed request to a machine learning model, determining if the request contains at least one critical symptom which corresponds to a critical illness based on an output of the machine learning model, assigning a weighted risk value to one or more general symptoms based on the output of the machine learning model, and determining if a sum of the weighted risk values for the one or more general symptoms exceeds a threshold value; recommend that the patient seek emergency medical services in response to determining that the request contains at least one critical symptom or determining that the sum of the weighted risk values exceeds the threshold value; and in response to determining the request does not contain at least one critical symptom and the sum of the weighted risk values does not exceed the threshold value, recommend a medical care provider to visit the patient based at least in part on the availability information of the medical care provider.

Clause 11. The system of clause 10, wherein the server is further configured to determine a confidence score associated with a recommendation that the medical care provider visit the patient.

Clause 12. The system of clause 10, wherein the server is further configured to receive expertise information of at least one medical care provider.

Clause 13. The system of clause 12, wherein the server is further configured to recommend the medical care provider based at least in part on the expertise information of the medical care provider.

Clause 14. The system of clause 10, wherein the availability information also includes cost information.

Clause 15. The system of clause 10, wherein the server is further configured to contact the emergency medical services based on a recommendation for the patient to seek emergency medical services.

Clause 16. The system of clause 10, wherein the request is received via a patient portal executing in a web browser.

Clause 17. The system of clause 10, wherein the availability information is received via a provider portal executing in a web browser.

Clause 18. The system of clause 10 wherein the one or more elements of the parsed request includes at least one of a location, a symptom, a medical history information, an indication of allergies, an indication of pain, and demographic information.

Clause 19. An apparatus for assisting with medical care dispatch comprising: means for receiving a request for a patient to receive medical care including, at least, patient symptoms and patient personal medical history; means for receiving availability information of at least one medical care provider; means for determining if the patient is experiencing a medical emergency based at least in part on the patient symptoms and the patient personal medical history; wherein the determining if the patient is experiencing the medical emergency includes parsing the request, providing one or more elements of the parsed request to a machine learning model, determining if the request contains at least one critical symptom which corresponds to a critical illness based on an output of the machine learning model, assigning a weighted risk value to one or more general symptoms based on the output of the machine learning model, and determining if a sum of the weighted risk values for the one or more general symptoms exceeds a threshold value; means for recommending that the patient seek emergency medical services in response to determining that the request contains at least one critical symptom or determining that the sum of the weighted risk values exceeds the threshold value; and means for recommending, in response to determining the request does not contain at least one critical symptom and the sum of the weighted risk values does not exceed the threshold value, a medical care provider to visit the patient based at least in part on the availability information of the medical care provider.

Clause 20. The apparatus of clause 19, further comprising means for determining a confidence score associated with a recommendation that the medical care provider visit the patient.

Clause 21. The apparatus of clause 19, further comprising means for receiving expertise information of at least one medical care provider.

Clause 22. The apparatus of clause 21, wherein the recommending the medical care provider is based at least in part on the expertise information of the medical care provider.

Clause 23. The apparatus of clause 19, wherein the availability information also includes cost information.

Clause 24. The apparatus of clause 19, further comprising means for contacting the emergency medical services in response to determining that the request contains at least one critical symptom or determining that the sum of the weighted risk values exceeds the threshold value.

Clause 25. The apparatus of clause 19, wherein the request is received via a patient portal executing in a web browser.

Clause 26. The apparatus of clause 19, wherein the availability information is received via a provider portal executing in a web browser.

Clause 27. The apparatus of clause 19 wherein the one or more elements of the parsed request includes at least one of a location, a symptom, a medical history information, an indication of allergies, an indication of pain, and demographic information.

Clause 28. A non-transitory processor-readable storage medium comprising processor-readable instructions configured to cause one or more processors to assist with medical care dispatch, comprising: code for receiving a request for a patient to receive medical care including, at least, patient symptoms and patient personal medical history; code for receiving availability information of at least one medical care provider; code for determining if the patient is experiencing a medical emergency based at least in part on the patient symptoms and the patient personal medical history; wherein the code for determining if the patient is experiencing the medical emergency includes code for parsing the request, code for providing one or more elements of the parsed request to a machine learning model, code for determining if the request contains at least one critical symptom which corresponds to a critical illness based on an output of the machine learning model, code for assigning a weighted risk value to one or more general symptoms based on the output of the machine learning model, and code for determining if a sum of the weighted risk values for the one or more general symptoms exceeds a threshold value; code for recommending that the patient seek emergency medical services in response to determining that the request contains at least one critical symptom or determining that the sum of the weighted risk values exceeds the threshold value; and code for recommending a medical care provider to visit the patient based at least in part on the availability information of the medical care provider in response to determining the request does not contain at least one critical symptom and the sum of the weighted risk values does not exceed the threshold value.

Clause 29. The non-transitory storage medium of clause 28, further comprising code for determining a confidence score associated with a recommendation that the medical care provider visit the patient.

Clause 30. The non-transitory storage medium of clause 28, further comprising code for receiving expertise information of at least one medical care provider.

Clause 31. The non-transitory storage medium of clause 30, further comprising code for recommending the medical care provider based at least in part on the expertise information of the medical care provider.

Clause 32. The non-transitory storage medium of clause 28, wherein the availability information also includes cost information.

Clause 33. The non-transitory storage medium of clause 28, further comprising code for contacting the emergency medical services.

Clause 34. The non-transitory storage medium of clause 28, wherein the request is received via a patient portal executing in a web browser.

Clause 35. The non-transitory storage medium of clause 28, wherein the availability information is received via a provider portal executing in a web browser.

Clause 36. The non-transitory storage medium of clause 28 wherein the one or more elements of the parsed request includes at least one of a location, a symptom, a medical history information, an indication of allergies, an indication of pain, and demographic information.

Clause 37. A method for anticipating demand for medical services, comprising: receiving, at a server, historical data of demand for medical services, including, at least, quantity data of medical service requests, geographical data of medical service requests, type data of medical service requests, determining, by the server, expected demand for medical services, including, at least, expected quantity of medical service requests, expected geographical locations of medical service requests, expected types of medical service requests, recommending, by the server, a quantity and geographical placement of medical care providers based, at least in part, on the expected demand for medical services.

1. A method for assisting with medical care dispatch, comprising:
receiving, at a server, a request for a patient to receive medical care including, at least, patient symptoms and patient personal medical history;
receiving, at the server, availability information of at least one medical care provider;
determining, by the server, if the patient is experiencing a medical emergency based at least in part on the patient symptoms and the patient personal medical history;
wherein the determining if the patient is experiencing the medical emergency includes parsing, by the server, the request,
providing one or more elements of the parsed request to a machine learning model,
determining if the request contains at least one critical symptom which corresponds to a critical illness based on an output of the machine learning model,
assigning a weighted risk value to one or more general symptoms based on the output of the machine learning model, and
determining if a sum of the weighted risk values for the one or more general symptoms exceeds a threshold value;
recommending, by the server, that the patient seek emergency medical services in response to determining that the request contains at least one critical symptom or determining that the sum of the weighted risk values exceeds the threshold value; and
in response to determining the request does not contain at least one critical symptom and the sum of the weighted risk values does not exceed the threshold value, recommending, by the server, a medical care provider to visit the patient based at least in part on the availability information of the medical care provider.

2. The method of claim 1, further comprising determining, by the server, a confidence score associated with a recommendation that the medical care provider visit the patient.

3. The method of claim 1, further comprising receiving, by the server, expertise information of at least one medical care provider.

4. The method of claim 3, wherein the recommending, by the server, the medical care provider is based at least in part on the expertise information of the medical care provider.

5. The method of claim 1, wherein the availability information also includes cost information.

6. The method of claim 1, wherein the recommending, by the server, that the patient seek emergency medical service further comprises contacting the emergency medical services.

7. The method of claim 1, wherein the request is received via a patient portal executing in a web browser.

8. The method of claim 1, further comprising recommending, by the server, to decline the request in response to determining, based on the availability information, that no medical care provider is available to treat the patient.

9. The method of claim 1 wherein the one or more elements of the parsed request includes at least one of a location, a symptom, a medical history information, an indication of allergies, an indication of pain, and demographic information.

10. A system for assisting with medical care dispatch comprising:
a server including a memory and at least one processor communicatively coupled to the memory and configured to:
receive a request for a patient to receive medical care including, at least, patient symptoms and patient personal medical history;
receive availability information of at least one medical care provider;
determine if the patient is experiencing a medical emergency based at least in part on the patient symptoms and the patient personal medical history;
wherein the determining if the patient is experiencing the medical emergency includes parsing the request,
providing one or more elements of the parsed request to a machine learning model,
determining if the request contains at least one critical symptom which corresponds to a critical illness based on an output of the machine learning model,
assigning a weighted risk value to one or more general symptoms based on the output of the machine learning model, and
determining if a sum of the weighted risk values for the one or more general symptoms exceeds a threshold value;
recommend that the patient seek emergency medical services in response to determining that the request contains at least one critical symptom or determining that the sum of the weighted risk values exceeds the threshold value; and
in response to determining the request does not contain at least one critical symptom and the sum of the weighted risk values does not exceed the threshold value, recommend a medical care provider to visit the patient based at least in part on the availability information of the medical care provider.

11. The system of claim 10, wherein the server is further configured to determine a confidence score associated with a recommendation that the medical care provider visit the patient.

12. The system of claim 10, wherein the server is further configured to receive expertise information of at least one medical care provider.

13. The system of claim 12, wherein the server is further configured to recommend the medical care provider based at least in part on the expertise information of the medical care provider.

14. The system of claim 10, wherein the availability information also includes cost information.

15. The system of claim 10, wherein the server is further configured to contact the emergency medical services based on a recommendation for the patient to seek emergency medical services.

16. The system of claim 10, wherein the request is received via a patient portal executing in a web browser.

17. The system of claim 10, further comprising recommending, by the server, to decline the request in response to determining, based on the availability information, that no medical care provider is available to treat the patient.

18. The system of claim 10 wherein the one or more elements of the parsed request includes at least one of a location, a symptom, a medical history information, an indication of allergies, an indication of pain, and demographic information.

19. An apparatus for assisting with medical care dispatch comprising:
means for receiving a request for a patient to receive medical care including, at least, patient symptoms and patient personal medical history;
means for receiving availability information of at least one medical care provider;
means for determining if the patient is experiencing a medical emergency based at least in part on the patient symptoms and the patient personal medical history;
wherein the determining if the patient is experiencing the medical emergency includes parsing the request,
providing one or more elements of the parsed request to a machine learning model,
determining if the request contains at least one critical symptom which corresponds to a critical illness based on an output of the machine learning model,
assigning a weighted risk value to one or more general symptoms based on the output of the machine learning model, and
determining if a sum of the weighted risk values for the one or more general symptoms exceeds a threshold value;
means for recommending that the patient seek emergency medical services in response to determining that the request contains at least one critical symptom or determining that the sum of the weighted risk values exceeds the threshold value; and
means for recommending, in response to determining the request does not contain at least one critical symptom and the sum of the weighted risk values does not exceed the threshold value, a medical care provider to visit the patient based at least in part on the availability information of the medical care provider.

20. The apparatus of claim 19, further comprising means for contacting the emergency medical services in response to determining that the request contains at least one critical symptom or determining that the sum of the weighted risk values exceeds the threshold value.
